(19) United States
(12) Patent Application Publication
(10) Pub. No.: US 2022/0111174 A1
(43) Pub. Date: Apr. 14, 2022

(54) THIN-WALLED TUBES WITH COMMUNICATION PATHWAYS

(71) Applicant: Zeus Industrial Products, Inc., Orangeburg, SC (US)

(72) Inventors: John Richard Campanelli, West Columbia, SC (US); Josh Fogle, Columbia, SC (US)

(21) Appl. No.: 17/555,834

(22) Filed: Dec. 20, 2021

Related U.S. Application Data
(63) Continuation of application No. PCT/US2020/039829, filed on Jun. 26, 2020.
(60) Provisional application No. 62/868,426, filed on Jun. 28, 2019.

(51) Int. Cl. A61M 25/00; C09D 11/52; A61L 29/08; A61L 29/14

(52) U.S. Cl. CPC A61M 25/0045; C09D 11/52; A61M 2205/0233; A61L 29/14; A61M 2025/0058; A61L 29/085

(57) ABSTRACT

The present disclosure provides modified polymeric thin-walled tubes with one or more conductive pathways along at least a part of a length or a circumference of the polymeric tube, suitable for use as liners in catheter construction. The one or more conductive pathways are formed of a conductive ink and are on a surface of the polymeric tube and not embedded within the walls of the tube.

### CROSS-REFERENCE TO RELATED APPLICATIONS

The present application is a continuation of PCT Application no. PCT/US2020/039829, filed Jun 26, 2020; which application claims the benefit of U.S. Provisional Application No. 62/868,426, filed Jun. 28, 2019. The disclosures of the aforementioned applications are incorporated herein by reference in their entirety.

### FIELD OF THE INVENTION

The present application relates generally to the field of catheters comprising thin wall catheter liners comprising poly(tetrafluoroethylene) (PTFE) and to methods relating to making and using such catheter liners and corresponding catheters.

### BACKGROUND OF THE INVENTION

A number of patents and applications teach the use of conductors to transmit electrical signals along a catheter shaft. For catheters with embedded sensors, communication pathways (e.g., conductive pathways) are required to relay signals (e.g., electrical signals (analog or digital)) from sensors or transducers at the distal end (e.g., force (digital or mechanical), tactile sensors (capacitive, piezoelectric, and strain), temperature, chemical, biological) to readouts at the proximal end and vice versa. However, with respect to communication pathways for electrical signals, electrical conductors that run through lumens in the catheter are inefficient. For example, such electrical conductors extending through such lumens take up cross-sectional area that could be used for instruments. In addition, such electrical conductors can increase the stiffness of the catheter to an unacceptable degree.

For example, U.S. Pat. No. 5,372,603 to Acker et al. generally describes copper conductors. The conductors of the '603 patent are not taught to maintain flexibility when depositing grains of conductive materials to produce the conductive pathway; rather, such conductors use an inelastic material as a substrate to prevent the cracking of the deposited grains of conductive materials.

U.S. Patent Application Publication No. 2008/0125754 to Beer et al. discloses a method to create conductors in a plastic tube by embedding an electrically conductive paste or slurry into a channel created by a focused energy beam. This approach requires a mounting step in a displaceable and rotatable mounting adapted to provide both axial and rotational motion subsequent to extrusion of the tube, adding cost and complexity to the overall manufacturing process and adversely affecting product yield. Further, the focused energy beam used to create the channel must remove material and such removal can be delicate and risky (especially for the production of thinner-wall liners (e.g., such as Zeus Streamliner® products, with wall thicknesses on the order of 0.019 mm). The removal of material from tubes having such small wall thicknesses is likely to decrease the yields of acceptable tubes for use in the manufacture/use of catheters. Additionally, unacceptable adhesion of the conductive paste or slurry to the newly created channel could reasonably result from such a process.

U.S. Patent Application Publication No. 2005/0165301 to Smith et al. describes electrically conductive paths in an MRI-compatible catheter but does not address how the path is created, nor how adhesion is maintained between the conductive trace and the surrounding polymers.

U.S. Patent Application Publication No. 2015/0173773 to Bowman et al. describes the use of a conductive trace running over the inner catheter liner with a hypotube then positioned over the trace. In this case the trace is secured in place with an adhesive, for example an epoxy, which would introduce added stiffness to the overall structure.

U.S. Pat. No. 9,554,723 to Anderson et al. discloses embedding electrically conductive wire or other conductive media within the walls of a polymeric tube to produce catheter shafts (including, e.g., within the wall of the inner tube). This approach can be particularly problematic, e.g., for liners with very thin walls as noted above.

International Patent Application Publication No. WO2014168987 to Salahieh et al. describe the construction of cardiac ablation catheters with electrodes made from a thin film of electro-conductive ink secured to the exterior of an expandable membrane. This disclosure does not teach the creation of conductive pathways directly along the catheter liner.

International Patent Application Publication No. WO2018035000 to Lowery et al. discloses the application of one or more electrically conductive tracings along the length of an elongate member for use in a medical device. This reference teaches depositing tracings on the outside surface of a tubular membrane and then everting the membrane to place the tracings in the everted inside surface, which may then be covered with a thin insulating/dielectric coating.

The use of conductive pathways such as inks and pastes generally requires embedding the materials into the tube wall, which is problematic for tubes having thin walls. There is a growing need for thinner, more flexible catheters and methods of producing such catheters for many types of minimally invasive interventions.

### SUMMARY OF THE INVENTION

The present disclosure provides modified polymeric tubes with ink-based conductive pathways thereon and methods for preparing such tubes which render the materials suitable, e.g., for use in inner wall (base liner) applications.

In one aspect of the disclosure is provided a modified polymeric tube suitable for use as a catheter liner, comprising: a polymeric tube with walls having an average wall thickness of about 0.5 mm or less; and one or more conductive pathways positioned directly on a surface of the polymeric tube or directly on an adhesive material in direct contact with the surface of the tube, along at least a part of a length or a circumference of the polymeric tube, wherein the one or more conductive pathways comprise a conductive ink and wherein the one or more conductive pathways are on a surface of the polymeric tube and not embedded within the walls of the polymeric tube. The tubes, in some embodiments, can withstand temperatures up to about 300° C.

In some embodiments, the one or more conductive pathways comprise a plurality of conductive pathways. In some embodiments, at least two of the plurality of conductive pathways are disposed to be at least electrically insulated with respect to each other. In some embodiments, an electrical insulation is disposed between the at least two of the plurality of conductive pathways. In some embodiments, the at least two of the plurality of conductive pathways are disposed at a same radial position of the polymeric tube and a different position around a circumference of the polymeric tube. In some embodiments, the at least two of the plurality of conductive pathways are disposed at a different radial position of the polymeric tube and a same position around a circumference of the polymeric tube. In some embodiments, an electrical insulation is disposed on at least a part of a length of the polymeric tube between the at least two of the plurality of conductive pathways.

The tubes can vary in average wall thickness. For example, in some embodiments, the average wall thickness is about 0.05 mm or less. In some embodiments, the average wall thickness is about 0.02 mm or less. In some embodiments, the surface of the tube comprises an outer surface of the tube. The composition of the tube can vary. In some embodiments, the tube comprises a polymer selected from the group consisting of PTFE, FEP, PFA, PEEK, UHMWPE, polyether block copolymers, polyamide block copolymers, polyether-polyamide copolymers, polyamides, polyimide, and co-polymers and derivatives thereof. Where the tube comprises an optional adhesive material, the material is, in some embodiments, polyimide.

In another aspect is provided a jacket-coated liner for use in a catheter, comprising a modified polymeric tube as provided herein and an outer jacket layer on the surface of the modified polymeric tube and directly adhered to the surface thereof. The conductive pathway of such jacket-coated liners advantageously exhibit conductivity. They may, in some embodiments, exhibit conductivity even when flexed. In some embodiments, the outer jacket layer comprises a nylon material or a poly(ether-b-amide). In a further aspect is provided a catheter comprising the modified polymeric tube or jacket-coated liner described herein.

In an additional aspect is provided method of providing a modified polymeric tube suitable for use as a catheter liner, comprising: providing a polymeric tube with walls having an average wall thickness of about 0.5 mm or less; and applying a conductive ink directly to a surface of the polymeric tube or on an adhesive material in direct contact with the surface of the polymeric tube in a pre-determined geometry, along at least a part of a length or a circumference of the polymeric tube to form conductive pathways on the polymeric tube, such that the conductive ink is not embedded within the walls of the polymeric tube. In some embodiments, the method further comprises treating the polymeric tube prior to applying the conductive ink. In some embodiments, the treating comprises chemically or physically treating the surface of the polymeric tube to enhance adhesion between the surface of the polymeric tube and the conductive ink. Applying can comprise, e.g., printing, electrostatic application, physical deposition, chemical deposition, vapor deposition, metallization, extrusion coating, and/or electroplating techniques.

In a still further aspect is provided a method of providing a jacket-coated liner for use in a catheter, comprising: providing a modified polymeric tube suitable for use as a catheter (as provided herein); melting a polymeric material on an outer surface of the modified polymeric tube; and cooling the polymeric material to give an outer jacket layer on the outer surface, wherein the outer jacket layer is directly adhered to the outer surface of the modified polymeric tube. In some embodiments, the polymeric material of the outer jacket layer comprises a nylon material or a poly(ether-b-amide).

The disclosure further provides, in an additional aspect, a method of forming a catheter, comprising: providing a jacket-coated liner for use in a catheter according to the methods disclosed herein; and incorporating the jacket-coated liner within a catheter assembly.

These and other features, aspects, and advantages of the disclosure will be apparent from a reading of the following detailed description together with the accompanying drawings, which are briefly described below. The invention includes any combination of two, three, four, or more of the above-noted embodiments as well as combinations of any two, three, four, or more features or elements set forth in this disclosure (e.g., in this Summary), regardless of whether such features or elements are expressly combined in a specific embodiment description herein. This disclosure is intended to be read holistically such that any separable features or elements of the disclosed invention, in any of its various aspects and embodiments, should be viewed as intended to be combinable unless the context clearly dictates otherwise. Other aspects and advantages of the present invention will become apparent from the following.

### BRIEF DESCRIPTION OF THE DRAWINGS

In order to provide an understanding of embodiments of the invention, reference is made to the appended drawing, which is not necessarily drawn to scale, and in which reference numerals refer to components of exemplary embodiments of the invention. The drawing is exemplary only, and should not be construed as limiting the invention.

FIGS. 1A-1H are schematic drawings of certain, non-limiting embodiments of tubes comprising ink-based conducting pathways;

FIGS. 2A and 2B are schematic drawing of one non-limiting embodiment of a tube comprising ink-based conducting pathways (with 2A providing a side view and 2B providing a cross-sectional view); and

FIG. 3 is a general schematic of one embodiment of a method provided herein for the construction of a tube comprising one or more conducting pathways.

### DETAILED DESCRIPTION OF THE INVENTION

The present invention now will be described more fully hereinafter. This invention may, however, be embodied in many different forms and should not be construed as limited to the embodiments set forth herein; rather, these embodiments are provided so that this disclosure will be thorough and complete, and will fully convey the scope of the invention to those skilled in the art. As used in this specification and the claims, the singular forms “a,” “an,” and “the” include plural referents unless the context clearly dictates otherwise.

The present invention is directed to tubes (e.g., thin-walled tubes) with conductive pathways comprising a conductive ink (also referred to herein as “ink-based conductive pathways”) and to methods of providing and using such tubes. Advantageously, in some embodiments, tubes provided herein with ink-based conductive pathways maintain flexibility, e.g., rendering them suitable for use in catheters, without causing delamination of the layers associated therewith. In particular, the conductive ink is advantageously printed directly on a surface of the tube to provide the conductive pathway(s) (or, as referenced below, on a thin adhesive material thereon to promote adhesion) rendering the disclosed principles suitable, e.g., for providing conductive pathways on very thin tubes/liners (where it is not possible to embed a conductive trace as commonly done in the field).

According to the present disclosure, tubes are provided comprising a conductive ink that has been applied to a surface of the tube (e.g., on an outer and/or surface thereof) to provide one or more conductive pathways. As used herein, “conductive pathways” refers to electrically conductive regions that are capable of carrying a current. The conductive ink serves as a pathway for signal transmission.

The conductive ink itself may serve as a sensing medium or be connected to a sensor and/or a signal receiver. In some embodiments, the ink-based conductive pathways are configured so as to allow signals to enter (e.g., at one end of a pathway) and pass therethrough to, e.g., a sensor/monitoring device (e.g., at an opposing end of a pathway). Conductive pathways may be configured with any number and type of additional elements (e.g., leads, electrical ports, etc.) to allow signals to effectively pass into, out of, and/or through the conductive pathways. “Tubes” are understood in their usual sense, to be elongated, cylindrical-shaped constructs of a given length L (from proximal end to distal end), with an outer surface and an inner surface defining a lumen. The diameter of the lumen is referred to as the tube's “inner diameter,” or “ID,” while the full diameter from outer surface to outer surface of the cross-section of the tube, across the lumen is referred to as the tube's “outer diameter,” or “OD.”

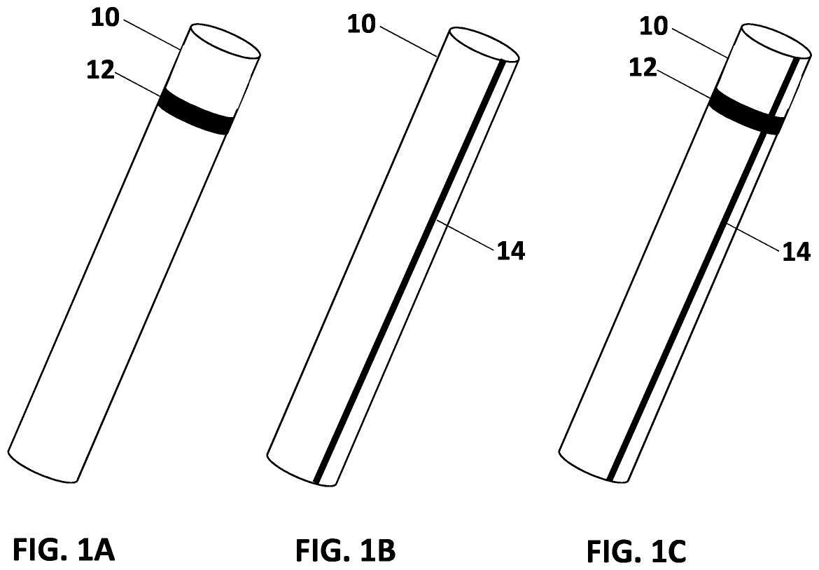

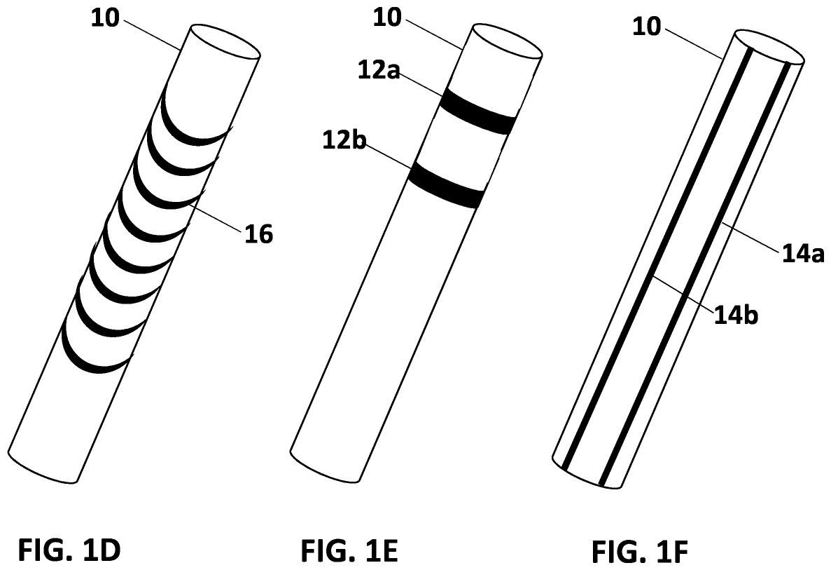

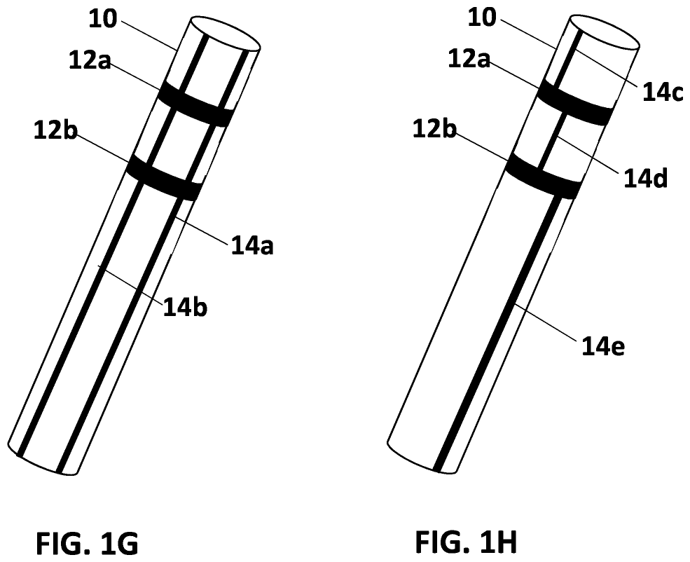

The ink-based conductive pathways may be on the inner and/or outer surfaces of the tube in a number of geometrical patterns including but not limited to fully coating the tube down the entire length, and/or applying single or numerous tracers over the length, centrifugally or in a spiral fashion. Certain, non-limiting patterns on outer surface of tubes are provided in FIGS. 1A-1F (leads and other associated sensors, etc. not shown). According to various embodiments, one or more ink-based conductive pathways are provided around at least a portion of the circumference of the tube or along at least a portion of the length of the tube. Simplified, non-limiting examples of these designs are provided in the tubes of FIGS. 1A and 1B, respectively, where 10 represents the tube, and 12 and 14 represents the one or more ink-based conductive pathways, respectively. FIG. 1C provides a non-limiting example embodiment of a tube comprising both an ink-based conductive pathway 12 around at least a portion of the circumference of the tube and an ink-based conductive pathway 14 along at least a portion of the length of the tube. It is noted that longitudinal pathways 14 need not extend the full length of the tube.

In some embodiments, they may extend only from one circumferential pathway 12 to another circumferential pathway 12. Circumferential pathways may wrap around the tube 10 so as to form a complete circle/band around the tube or may be spiral-shaped, wrapping in a coiled fashion around the tube 10 (as shown in FIG. 1D, comprising spiral ink-based conductive pathway 16). In some embodiments, tubes are provided with two or more ink-based conductive pathways. In some embodiments, at least two of the one or more conductive pathways are spaced apart from another, as shown in FIG. 1E (pathways 12a and 12b spaced apart), FIG. 1F (pathways 14a and 14b spaced apart), and FIG. 1G (pathways 12a and 12b and 14a and 14b spaced apart). As example, such space or material of the tube between any two conductive pathways can serve as an insulator between such two conductive pathways. In some embodiments, at least two of the one or more conductive pathways are spaced apart from another by disposing such one or more conductive pathways at different locations along the circumference of the tube, as shown, e.g., in FIGS. 1F and 1G).

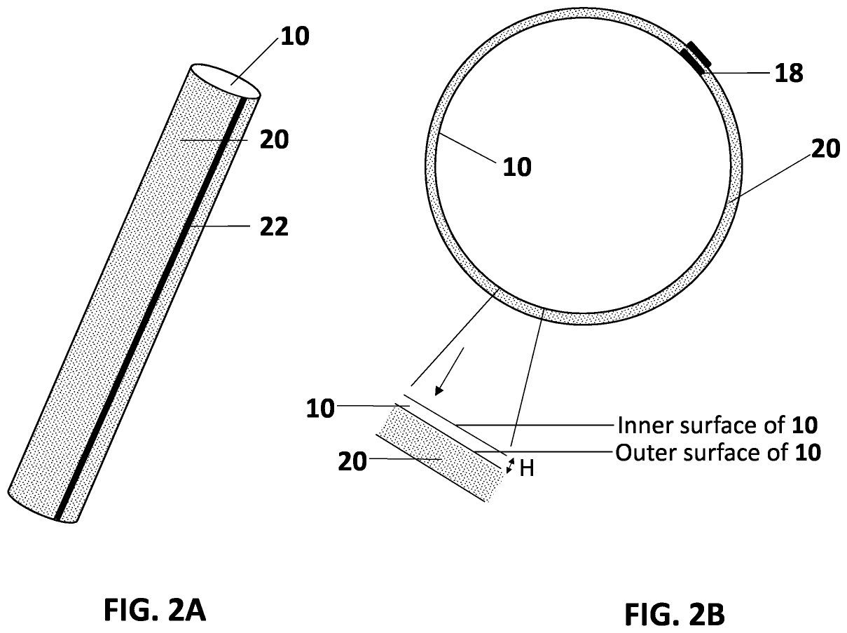

In some embodiments, at least two of the one or more conductive pathways are spaced apart by an insulating layer being disposed there between. For example, as depicted FIGS. 2A and 2B, a first conductive pathway 18 (shown in black) can be created along an outer length of the tube 10 at a first radial location, an insulating layer 20 (shown as black dots on white) can be created (e.g., disposed) over the first conductive pathway 18 along the outer length of the tube at such first radial location, and a second conductive pathway 22 (shown in black) can be created along the outer length of the tube (e.g., over the first insulating layer) at such first radial location. As shown in FIG. 2A, only the second conductive pathway 22 is visible on the surface of the tube. The insulating layer may cover the entirety of the tube (as shown) or may cover just a portion of the tube, e.g., a portion suitable to cover the first conductive pathway 18. FIG. 2B includes a magnified portion of a portion of the tube without a conductive pathway thereon, showing the inner and outer surfaces of tube 10 (with distance from inner to outer surface shown as average thickness, “T”), and the insulating coating 20 thereon) (layers not necessarily drawn to scale).

The sizes and shapes of the conductive pathways can vary. In some embodiments, a conductive pathway is configured based at least in part on a signal to be communicated or measured over the conductive pathway. For example, impurities (insulators) can be introduced to the conductive pathway which will provide an impedance or resistance along the conductive pathway where desired. In some embodiments, a conductive pathway is configured based at least in part on a signal to be communicated over the conductive pathway. For example, a material or size or shape of the conductive pathway can be configured based at least in part on the signal to be communicated. In other embodiments, a conductive pathway is configured based at least in part on a signal to be measured over the conductive pathway. For example, a material or size or shape of the conductive pathway can be configured based at least in part on the signal to be measured. In another embodiment, the conductive pathway can be used at least in part to generate a signal from an interaction with the immediate environment of the tube. The interaction could be, for example, chemical, physical or biological in nature, and could occur anywhere along the length of the pathway. In some embodiments, a conductive pathway is configured based at least in part on a signal to be generated/measured over the conductive pathway and the longitudinal stiffness of the tube. For example, a material or size or shape of the conductive pathway can be configured based at least in part on the stiffness desired between proximal and distal end. Typical thicknesses of the ink-based conductive pathways, where present on a tube surface, are generally low (described as the average distance/height extending radially outward from the outer surface of the tube). In certain embodiments, average thicknesses relative to the tube surface of the disclosed ink-based conductive pathways are in the range of about 10 microns or less. In some embodiments, it is advantageous to minimize the thickness of the ink-based conductive pathways to values within the above-mentioned range, e.g., as the thicker the layer, the more pronounced will be the I-beam effect for flexure around the centerline of the tube. The I-beam effect in this context is understood to mean the increase in overall stiffness of a catheter assembly around the centerline due to stiffer components being spatially located away from the centerline rather than close to the centerline. In order to maximize flexibility of the overall catheter assembly, it is desirable to locate stiffer components such as an electrically conductive layer as close as possible to the centerline of the catheter. Advantageously, the presently disclosed tubes can comprise ink-based conductive pathways on the outer surface of the innermost tube/liner to be used in a catheter assembly to minimize the I-beam effect. Moreover, by keeping the thickness of the ink-based conductive pathways low, flexibilities of tubes with such ink-based conductive pathways can be maintained, e.g., close to their original flexibilities. Advantageously, such tubes comprising ink-based conductive pathway exhibit flexibility suitable to render them particularly useful, e.g., in catheter constructions.

Tubes relevant in the context of the present disclosure can vary significantly in terms of size, shape, and composition. Similarly, ink materials that are suitable for use as the ink-based conductive pathways can vary, as described below.

The composition of the disclosed tubes is not particularly limited. In some embodiments, the tubes comprise materials suitable for use as catheter liners (rendering the resulting tube comprising ink-based conductive pathway(s) suitable for such use). Some tubes can comprise fluoropolymers, polyamides, polyimides, olefins, polyesters, and/or elastomers. This also includes copolymers and polymer blends (e.g., including, but not limited to, polyether block copolymers, polyamide block copolymers, and polyether-polyamide copolymers). Non-limiting examples of tube compositions include, but are not limited to, polymers comprising Pebax® (a block copolymer comprising polyamide and polyether blocks, commonly referred to as poly(ether-b-amide)), polyimide (PI), fluorinated ethylene propylene (FEP), perfluoroalkoxyalkane (PFA), polyether ether ketone (PEEK), ultra-high molecular weight polyethylene (UHMWPE), and copolymers and derivatives thereof. In another embodiment, the tube comprises poly(tetrafluoroethylene) (PTFE). One exemplary, non-limiting PTFE tube is Zeus Inc.'s Streamliner® products. Tubes can be homogenous or non-homogeneous. In some embodiments, tubes may comprise a coating on the inner and/or outer surface thereof.

As referenced above, the tubes provided herein can have relatively low wall thicknesses in some embodiments (although the principles have applicability to thicker-walled tubes as well). In some embodiments, the tubes herein have average wall thicknesses of about 0.5 mm or less, about 0.4 mm or less, about 0.3 mm or less, about 0.2 mm or less, about 0.1 mm or less, about 0.09 mm or less, about 0.08 mm or less, about 0.07 mm or less, about 0.06 mm or less, or about 0.05 mm or less. In some embodiments, the tubes provided herein have average wall thicknesses considered “ultrathin,” e.g., about 0.002″ or less, including about 0.001″ or less, about 0.0009″ or less, or about 0.0008″ or less (including, specifically, tubes with walls having an average thickness of about 0.00075″), i.e., about 0.051 mm or less, including about 0.025 mm or less, about 0.023 mm or less, or about 0.020 mm or less (including, specifically, tubes with walls having an average thickness of about 0.019 mm).

The ink chemistry can be selected to adhere to any of the types of polymer classes referenced above with respect to tube composition. In some embodiments, the ink is a conductive silver-containing ink. Some such inks comprise silver nanoparticles. In some embodiments, the ink is a conductive carbon-containing ink. Other polymer-based inks and metal-based inks (e.g., comprising tantalum, tungsten, gold, platinum, palladium, and alloys thereof) are known and can be employed in the context of the present disclosure. Certain non-limiting inks that meet the above requirements and are suitable for the disclosed products and methods can be obtained, for example, from Conductive Technologies Incorporated in York, Pa. Selection of an appropriate ink for a given type of tube can be done, e.g., based on hydrophilic/hydrophobic compatibility between the binder of the ink and the composition of the tube.

Since the conductive pathways are not embedded into the tube walls, adhesion of the conductive ink to the surface (e.g., outer surface) of the tube/liner and to any material applied over the pathway-containing tube (e.g., an outer jacket material) is very important. There are various ways to evaluate suitable adhesion of the ink to the tube, including, e.g., a “tape test.” A tape test can be conducted, e.g., according to known methods such as those in ASTM D3359-17. Although this test method allows for some degree of removal of ink following removal of the tape, in preferred embodiments of the present disclosure, no removal of ink is observed following removal of the tape, evidencing “suitable adhesion” according to the present disclosure.

The disclosed ink-based conductive pathways advantageously exhibit sufficient adhesion to a variety of substrates and, surprisingly are able to withstand catheter construction procedures, including braiding and reflow of an outer jacket material (e.g., including, but not limited to, a material comprising nylon and/or a polyetheramide block copolymer, e.g., Pebax®, i.e., poly(ether-b-amide)). Advantageously, the conductivity of the conductive pathways as-formed (before an outer jacket material is reflowed there over) is retained following the reflowing of an outer jacket material at elevated temperature. In some embodiments, the tube comprising ink-based conductive pathways is advantageously stable (e.g., retains conductivity properties) up to temperatures sufficient for this reflowing. Such temperatures at which the tubes are desirably stable depend on the temperature required for flow of the outer jacket material and include, but are not limited to, temperatures of about 300° C. or less, about 290° C. or less, about 280° C. or less, about 270° C. or less, about 260° C. or less. As such, the disclosed coated tubes can, in some embodiments, withstand application of such temperatures (i.e., up to and including about 300° C., about 290° C., about 280° C., about 270° C., or about 260° C.), and maintain conductivity following such exposure to heat.

The ink-based conductive pathway-modified tube provided herein is stable to high degrees of flex, maintaining conductivity properties even with significant bending/flex and/or exhibiting little to no delamination of layers associated with the ink-based conductive pathway-modified tube. Where the disclosed tubes comprising ink-based conductive pathways are incorporated within catheters, good adhesion is noted between various catheter layers (e.g., including between the conductive ink pathways and the tube, and between the tube/conductive ink pathways and the overlying outer jacket material). Advantageously for such purposes, adhesion between such layers is maintained during use of the catheter, such that even when the catheter assembly is flexed (e.g., to pass through tortuous body lumens), no delamination is observed, in particular, between the tube comprising the ink-based conductive pathways and any other adjacent layer. Advantageously, the presence of the conductive pathway(s) described herein on the tube surface does not negatively impact, to any significant extent, the ability of that tube surface to be suitably adhered to another adjacent layer (e.g., a jacket layer as referenced above). Suitable adhesion can be determined by visual observation, e.g., such that no delamination is observed.

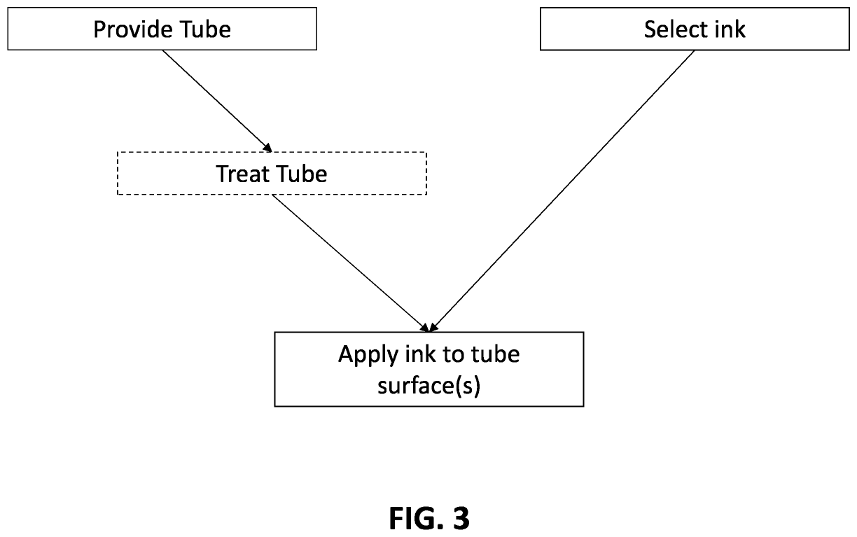

A general schematic of a method provided herein for the production of a conductive pathway on a tube is provided in FIG. 3. As shown therein, the method generally comprises providing a tube; optionally treating the tube, selecting an appropriate ink based on the tube composition; and applying the ink to desired regions of the tube.

The step of providing a tube can involve simply selecting the tube to be processed via the application of ink-based conductive pathways. As referenced herein above, the disclosed method is applicable to a range of tube sizes, shapes, wall thicknesses, and compositions. Selection of the tube may determine whether further treatment is required prior to applying conductive ink thereto. In particular, where the tube comprises a fluoropolymer, treatment is generally conducted prior to applying conductive ink thereto, e.g., to ensure suitable adhesion. Where the tube comprises a polymer other than a fluoropolymer, such treatment is typically not required to ensure sufficient adhesion. As such, in some embodiments, a tube can be used “as-is,” i.e., no further processing is necessary before applying the ink. However, the disclosed methods are not so limited, and such tubes may, in some embodiments, still be treated prior to applying conductive ink thereto.

The type of treating done can vary and typically provides for greater adhesion between the surface of the tube and the conductive ink to be applied. In some non-limiting embodiments, the treating makes the surface of the tube more hydrophilic. Such treating typically does not comprise physically altering the surface to any significant extent (e.g., not creating a channel for the formation of conductive pathways or embedding anything within the walls of the tubes). Treating can, in some embodiments, alter (e.g., increase) the surface roughness. For example, in some embodiments, the treating is physical treating (e.g., including, but not limited to, methods such as plasma treatment or electrical discharge). In some embodiments, the treating involves chemical treating. Chemical treating can involve, e.g., chemical etching as known in the art. In the context of, e.g., PTFE tube surfaces, chemical treating can involve treating with reagents that strip fluorine from the PTFE (replacing them with reactive groups). In other embodiments, the treatment comprises applying an adhesive layer to at least a portion of the surface to which the conductive ink will be applied. Suitable adhesives do not significantly affect the flexibility of the underlying tube. In some embodiments, the adhesives are not epoxy-based. In one embodiment, the adhesive is polyimide.

The method further comprises selecting an appropriate conductive ink for adhesion to a given type of polymeric tube (e.g., exhibiting suitable adhesion to the surface to which it will be applied, which can be the material of the tube itself, material of a chemically treated surface of the tube, or adhesive material on the surface of the tube. Types of conductive ink are referenced herein above and, as referenced above, can be selected based on anticipated compatibility between the surface and the ink.

The selected conductive ink is then applied to the desired portion(s) of the tube surface(s). The conductive ink is applied so as to provide any coating geometry/pattern, including those specifically referenced above. As such, the application is typically done in a controlled manner, so as to provide specific conductive pathways on the surface(s) of the tube. Advantageously, the methods provided herein do not require the creation of a channel, indentation, or any other type of mechanical indentation in the tube.

The method by which the ink is applied can vary. Exemplary methods suitable for this purpose include, but are not limited to, printing, electrostatic application, physical deposition, chemical deposition, vapor deposition, metallization, extrusion coating, and/or electroplating techniques. The conductive ink will have a low resistance therefore lessening the impedance or resistance of signal transmission and signal measurement. Alternatively, the conductive ink can be applied by extrusion-coating the conductive layer directly onto the tube. Insulating adhesive layers may, in some embodiments, be applied (e.g., coextruded) in subsequent operations so that multiple conductive channels are created. These processes can be repeated as needed to provide the desired geometry.

It will be immediately apparent to one skilled in the art that the tube of the invention does not have to be limited in size, e.g., length, to a size/length typically used for catheters, but that it can be produced in sizes/lengths suited to a number of other applications requiring tubes (including thin-walled tubes) such as in the telecommunications, aerospace, automotive and energy exploration sectors.

### Experimentals

Aspects of the present invention are more fully illustrated by the following examples, which are set forth to illustrate certain aspects of the present invention but are not be construed as limiting thereof.

### EXAMPLE 1

A silver-based conductive ink from Conductive Technologies Inc. with a width of 0.35 mm was applied down the length of a 0.5 m long Polyimide tube having an OD of 1.5 mm and a wall thickness of 0.125 mm. The resistance per foot was measured by the following method.

A Keithley 2100 6½ Digit Digital Multimeter capable of measuring 100 μΩ was used to measure the conductor resistance of the ink coating in the longitudinal direction for the Polyimide sample. Unicable probe style test leads were used to gather resistance measurements. Before testing coated samples, lead resistance was first measured. The resistance of the conductive pathway was then measured for a 610 mm length. Lead resistance was then taken into consideration and subtracted from the conductive pathway measurements yielding a reading of 29Ω.

Mandrel bend resistance was measured on the same sample mentioned previously by wrapping five full turns of the coated tubing around a 25 mm diameter mandrel. The coiled resistance of the coated tube measured 37Ω.

An adhesive tape with a known adhesion of 7 N/25 mm to steel was used to check the adhesion of the ink to the Polyimide substrate. A 76 mm length of the adhesive tape was placed with its center over the printed ink and smoothed. A “cure” time of 90±30 secs was allowed, after which the tape was removed rapidly. The removed adhesive tape presented with no signs of ink removal from the substrate, therefore showing excellent adhesion between the ink and the substrate.

### EXAMPLE 2

The tube of Example 1 was reflowed with a Pebax jacket and FEP heatshrink under the conditions described below.
Rate: 305 mm/min
Temperature: 260° C.

After reflow, the resistance of a 305 mm sample was measured to be 12Ω, indicating that the integrity of the conductive pathway had been maintained during the reflow process.

### EXAMPLE 3

A silver-based conductive ink from Conductive Technologies Inc. with a width of 0.25 mm was applied down the length of a 0.5 m long Pebax tube having an OD of 1.6 mm and a wall thickness of 0.145 mm.

A Keithley 2100 6½ Digit Digital Multimeter capable of measuring 100 μΩ was used to measure the conductor resistance of the ink coating in the longitudinal direction for the Pebax sample. Unicable probe style test leads were used to gather resistance measurements. Before testing coated samples, lead resistance was first measured. The resistance of the conductive pathway was then measured for a 610 mm length. Lead resistance was then taken into consideration and subtracted from the conductive pathway measurements yielding a reading of 31Ω.

Mandrel bend resistance was measured on the same sample mentioned previously by wrapping five full turns of the coated tubing around a 25 mm diameter mandrel. The coiled resistance of the coated tube measured 31Ω.

An adhesive tape with a known adhesion of 7 N/25mm to steel was used to check the adhesion of the ink to the Pebax substrate. A 76 mm length of the adhesive tape was placed with its center over the printed ink and smoothed. A “cure” time of 90±30 secs was allowed, after which the tape was removed rapidly. The removed adhesive tape presented with no signs of ink removal from the substrate, therefore showing excellent adhesion.

A second test to determine adhesion of the ink to the Pebax substrate was measured per modified ASTM D3359-17 using Method A X-Cut with the adhesive tape mentioned in the previous adhesion test. Cuts were made through the ink to the substrate for a 25 mm length. A 76 mm length of adhesive tape was then placed with its center over the scored area and smoothed. A “cure” time of 90±30 secs was allowed, after which the tape was removed rapidly. The removed adhesive tape presented with no signs of peeling or removal of the printed area, therefore showing excellent adhesion.

Many modifications and other embodiments of the invention will come to mind to one skilled in the art to which this invention pertains having the benefit of the teachings presented in the foregoing description. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

What is claimed is:

1. A modified polymeric tube suitable for use as a catheter liner, comprising:
a polymeric tube with walls having an average wall thickness of about 0.5 mm or less; and
one or more conductive pathways positioned directly on a surface of the polymeric tube or directly on an adhesive material in direct contact with the surface of the tube, along at least a part of a length or a circumference of the polymeric tube,
wherein the one or more conductive pathways comprise a conductive ink and
wherein the one or more conductive pathways are on a surface of the polymeric tube and not embedded within the walls of the polymeric tube.

2. The modified polymeric tube of claim 1, wherein the one or more conductive pathways comprise a plurality of conductive pathways.

3. The modified polymeric tube of claim 2, wherein at least two of the plurality of conductive pathways are disposed to be at least electrically insulated with respect to each other.

4. The modified polymeric tube of claim 3, wherein an electrical insulation is disposed between the at least two of the plurality of conductive pathways.

5. The modified polymeric tube of claim 4, wherein the at least two of the plurality of conductive pathways are disposed at a same radial position of the polymeric tube and a different position around a circumference of the polymeric tube.

6. The modified polymeric tube of claim 4, wherein the at least two of the plurality of conductive pathways are disposed at a different radial position of the polymeric tube and a same position around a circumference of the polymeric tube.

7. The modified polymeric tube of claim 3, wherein an electrical insulation is disposed on at least a part of a length of the polymeric tube between the at least two of the plurality of conductive pathways.

8. The modified polymeric tube of claim 1, wherein the average wall thickness is about 0.05 mm or less.

9. The modified polymeric tube of claim 1, wherein the average wall thickness is about 0.02 mm or less.

10. The modified polymeric tube of claim 1, wherein the surface of the tube comprises an outer surface of the tube.

11. The modified polymeric tube of claim 1, wherein the tube comprises a polymer selected from the group consisting of PTFE, FEP, PFA, PEEK, UHMWPE, polyether block copolymers, polyamide block copolymers, polyether-polyamide copolymers, polyamides, polyimide, and co-polymers and derivatives thereof.

12. The modified polymeric tube of claim 1, wherein the optional adhesive material is polyimide.

13. The modified polymeric tube of claim 1, wherein the modified tube can withstand temperatures up to about 300° C.

14. A jacket-coated liner for use in a catheter, comprising the modified polymeric tube of claim 1 and an outer jacket layer on the surface of the modified polymeric tube and directly adhered to the surface thereof.

15. The jacket-coated liner of claim 14, wherein the conductive pathways exhibit conductivity.

16. The jacket-coated liner of claim 14, wherein the outer jacket layer comprises a nylon material or a poly(ether-b-amide).

17. A catheter comprising the modified polymeric tube of claim 1.

18. A catheter comprising the jacket-coated liner of claim 14.

19. A method of providing a modified polymeric tube suitable for use as a catheter liner, comprising:
providing a polymeric tube with walls having an average wall thickness of about 0.5 mm or less; and
applying a conductive ink directly to a surface of the polymeric tube or on an adhesive material in direct contact with the surface of the polymeric tube in a pre-determined geometry, along at least a part of a length or a circumference of the polymeric tube to form conductive pathways on the polymeric tube, such that the conductive ink is not embedded within the walls of the polymeric tube.

20. The method of claim 19, wherein the method further comprises treating the polymeric tube prior to applying the conductive ink.

21. The method of claim 20, wherein the treating comprises chemically or physically treating the surface of the polymeric tube to enhance adhesion between the surface of the polymeric tube and the conductive ink.

22. The method of claim 19, wherein the applying comprises printing, electrostatic application, physical deposition, chemical deposition, vapor deposition, metallization, extrusion coating, and/or electroplating techniques.

23. A method of providing a jacket-coated liner for use in a catheter, comprising:
providing a modified polymeric tube suitable for use as a catheter liner according to the method of claim 19; and
melting a polymeric material on an outer surface of the modified polymeric tube and cooling the polymeric material to give an outer jacket layer on the outer surface, wherein the outer jacket layer is directly adhered to the outer surface of the modified polymeric tube.

24. The method of claim 23, wherein the polymeric material of the outer jacket layer comprises a nylon or a poly(ether-b-amide).

25. A method of forming a catheter, comprising:
providing a jacket-coated liner for use in a catheter according to the method of claim 23; and
incorporating the jacket-coated liner within a catheter assembly.
